(19) United States
(12) Patent Application Publication
(10) Pub. No.: US 2007/0038239 A1
(43) Pub. Date: Feb. 15, 2007

(54) Asymmetrically inflating flexi-tip gastroplasty calibration tube

(76) Inventor: James Dundas Ritchie, Edgecliffe (AU)

Correspondence Address: BROOKS KUSHMAN P.C., 1000 TOWN CENTER, TWENTY-SECOND FLOOR, SOUTHFIELD, MI, 48075, US

(21) Appl. No.: 10/555,149

(22) PCT Filed: May 27, 2004
(86) PCT No.: PCT/AU04/00708

§ 371 Date: Nov. 2, 2005

(30) Foreign Application Priority Data
May 27, 2003 (AU) 2003902600

(51) Int. Cl. A61M 29/00

(52) U.S. Cl. 606/192

(57) ABSTRACT

A gastric band positioning tubular apparatus including an extended tube (4) having a series of lumens (11, 12) therein; an asymmetrically inflating balloon (3) located substantially at a distal end of the extended tube and interconnected with one of said series of lumens for the application of a positive air pressure thereto for asymmetric inflation of said balloon.

### FIELD OF THE INVENTION

The present invention relates to the field of a gastroplasty tube utilisation during the insertion of an adjustable silicone gastric band.

### BACKGROUND OF THE INVENTION

Morbid obesity is a significant problem in modem western society. Morbid obesity occurs when the obesity is sufficient to prevent normal activity or physiologic function and is sufficient to cause onset of a pathological condition. People who are morbidly obese are more than 10 times as likely to die each year. In addition to the health risks, the socio-economic and psychological effects of morbid obesity are significant.

One of the standard methods of treatment of morbid obesity is the placement of a gastric band around the top portion of the stomach so as to limit the intake of foods. Adjustable gastric band devices are well known. Illustrations of gastric band devices can be found in U.S. Pat. Nos. 4,592,339, 6,694,982, 6,547,801, 6,676,674 and 5,549,621. The gastric banding procedure has been described in the medical journal articles by Solhan, “Gastric Banding: A New Method for Treatment of Morbid Obesity” Current Surgery pages 424-428, November-December 1983.

### SUMMARY OF THE INVENTION

It is an object of the present invention to provide for an improved form of gastric band positioning device.

In accordance with a first aspect of the present invention, there is provided: a gastric band positioning tubular apparatus including comprising an extended tube having a series of lumens therein; an asymmetrically inflating balloon located substantially at a distal end of the extended tube and interconnected with one of said series of lumens for the application of a positive air pressure thereto for asymmetric inflation of said balloon.

The extended tube can include a resilient flexible tip located at the distal end thereof. The resilient flexible tip can include a first neck portion and a second bulbous end portion interconnected to said neck portion. The tube can include a series of position markers along an exterior surface of the tube. The asymmetrically inflated tube can be located so as to inflate asymmetrically relative to said position markers.

A series of holes can be located in the distal end thereof, the holes interconnected to a second suction lumen for sucking air and gastric juices out of a patient's stomach in use. The tube can be formed from substantially from 80 Shore A hardness silicone. The balloon can be formed from substantially 30 Shore A Hardness silicone and can include a series of longitudinal ribbed portions on an exterior surface thereof and include a series of exterior markings for circumferentially positioning said balloon on said extended tube.

In accordance with another aspect of the present invention there is provided an asymmetrically inflating balloon for use gastric banding operations. The balloon can be formed from substantially 30 Shore A Hardness silicone and can include a series of longitudinal ribbed portions on an exterior surface thereof and include a series of exterior markings for circumferentially positioning said balloon on said extended tube.

In accordance with a further aspect of the present invention, there is provided a method of placing a gastric band, the method including: utilising an asymmetrically inflating balloon in the stomach to position the gastric band around the stomach.

### BRIEF DESCRIPTION OF THE DRAWINGS

Preferred forms of the present invention will now be described with reference to the accompanying drawings in which:

FIG. 1 illustrates schematically the operation of a calibration tube in accordance with gastric banding;

FIG. 2 illustrates a sectional view through the calibration tube of the preferred embodiment;

FIG. 3 illustrates a transfer section view through the arrangement of FIG. 2;

FIG. 4 and FIG. 5 illustrate side plane views of the arrangement of the preferred embodiment.

FIG. 6 is a side perspective view of an asymmetrical balloon.

### DESCRIPTION OF PREFERRED AND OTHER EMBODIMENTS

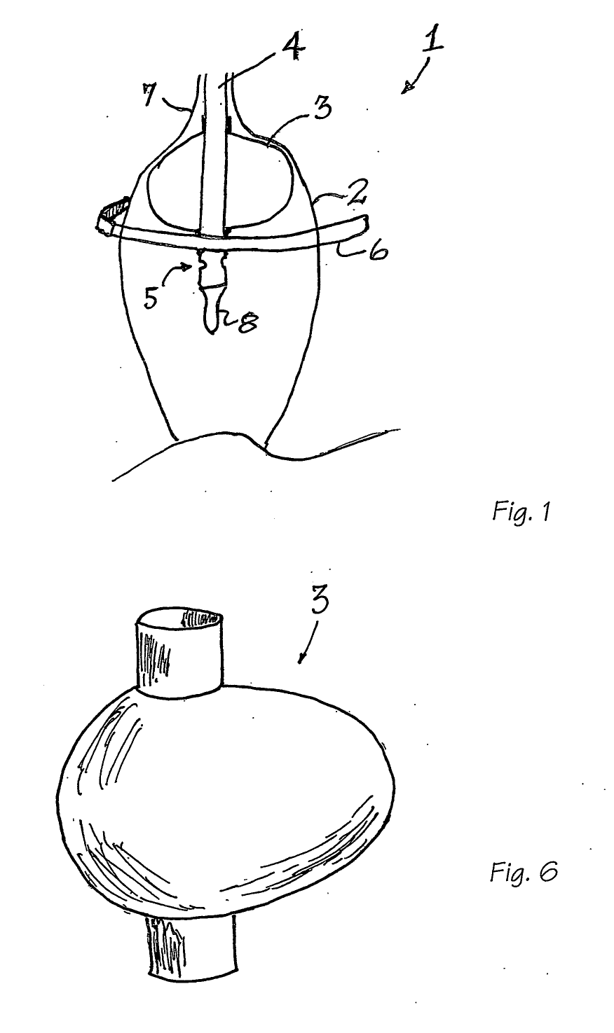

In the preferred embodiment, there is provided an asymmetrically inflating flexible tip gastroplasty calibration tube. Turning initially to FIG. 1, the preferred embodiment is designed for utilisation during gastric banding operations wherein the stomach wall 2 is appropriately profiled by means of an asymmetric balloon 3 which is activated via a calibration tube 4, the ends of which include a series of holes 5 for extraction of gastric juices from the stomach. The arrangement 1 is utilised in sizing the gastric pouch at the time of insertion of an adjustable silicone gastric band around the stomach 2. The insertion of gastric bands is well known and is described in the aforementioned patent specification. The balloon device 3 is designed to inflate asymmetrically so that it inflates substantially on the ventral side only. The balloon 3 is normally inflated between the gastric band 6 and the cardio oesophageal junction 7 during the surgical procedure to size and shape the pouch. The calibration tube also includes a flexible low trauma tip 8 for insertion. A series of suction holes e.g. 5 act to decompress the stomach by draining air and gastric juices from the stomach.

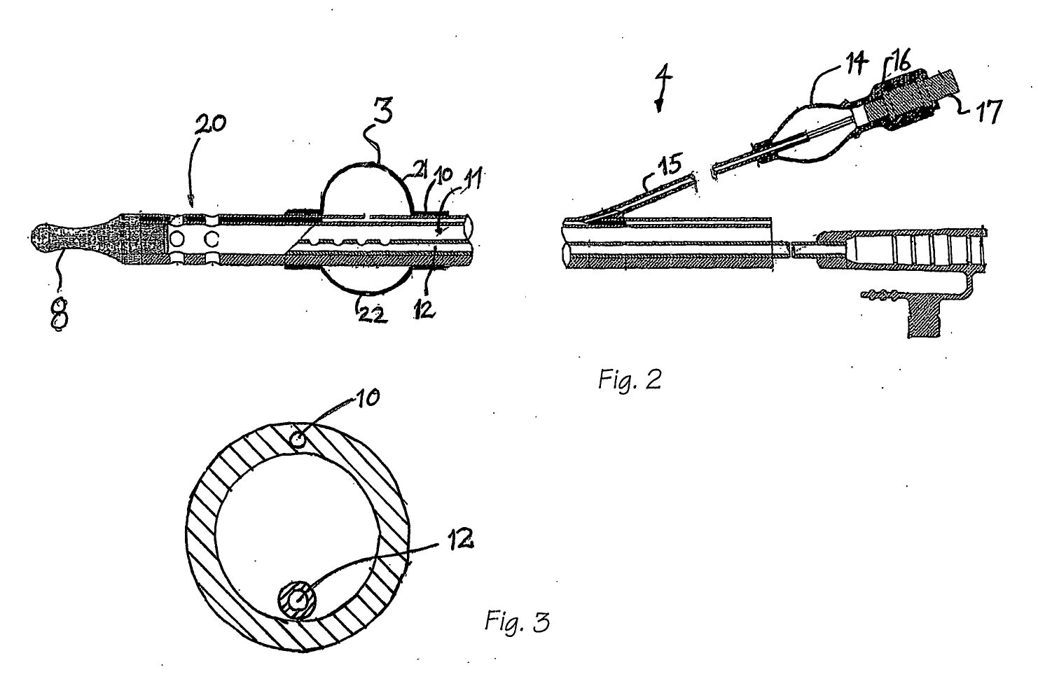

The calibration tube 4 is illustrated in more detail in FIG. 2. FIG. 2 illustrates a sectional view of the tube, which includes a number of internal lumens 10, 11, 12. The tube 4 can be formed to be 74 cms long with a 3 cm long silicon tip 8. The first lumen 10 is responsible for receiving a positive air pressure from a Medical Check Valve 14 with the pressure being communicated so as to inflate the balloon 3.

The lumen 10 is illustrated more clearly in FIG. 3 which is a sectional view through the main body of the calibration tube 4. A second suction tube lumen 12 is provided for the suction and removal of air and gastric juices. At the proximal end of the tube 4, the balloon inflation lumen 10 leads to a short external tube 15 which is connected to a medical check valve 16 equipped with a female luer taper fitting. This fitting 17 mates with a syringe which can be used to inflate the balloon with approximately 15-40 millilitres of air depending on the surgeons preferences. The distal end of the tube 4 between the balloon 3 and silicone tip 8 is a multi-perforated tube portion 20 which allows for efficient gastric decompression. The tip of the tube 8 includes a solid soft silicone rubber tip with a final bulbous tip and a flexible neck. This form of tip has been found to effectively safely pass through kinks and bends in the oesophagus and minimise the risk of perforation. The balloon 3 is eccentrically inflating. The side 21 inflates more readily than the side 22.

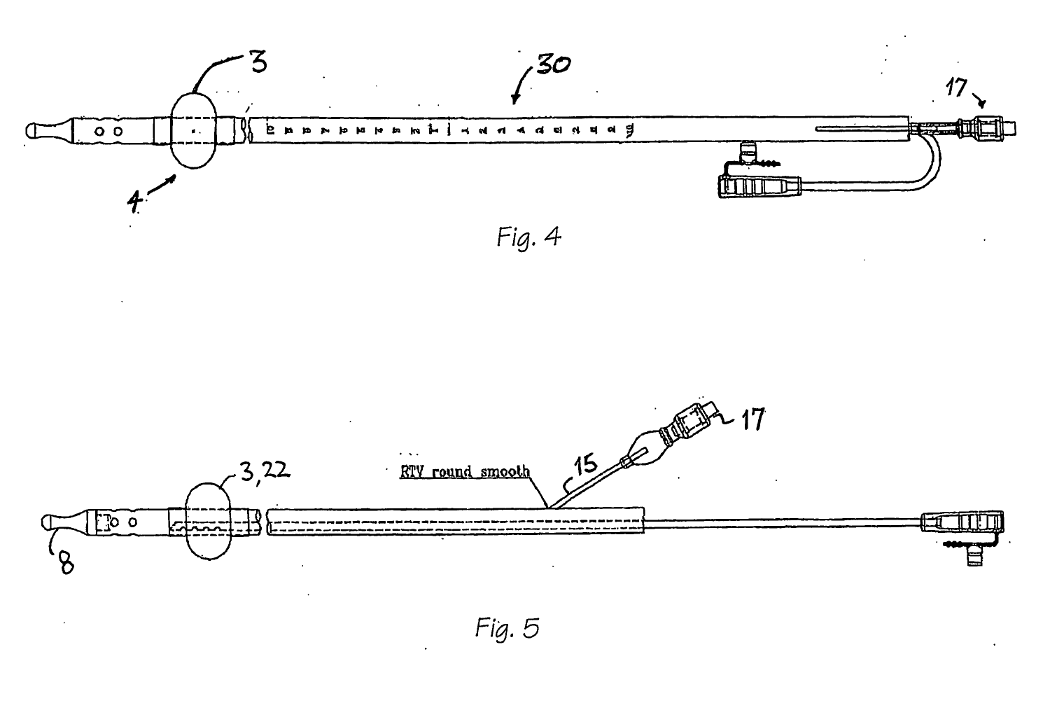

As illustrated in FIG. 4, the shaft of the tube carried a series of distance markers 30 which are intended to assist the anaesthetist to position the tube as requested by the surgeon.

The material used in the construction of the tube is preferably silicone. This material is considered suitable as it produces less friction and therefore potentially less friction trauma to patient's oesophagus and stomach. Silicone material also does not produce chemical reactions with the gastric juices, and its flexibility allows the catheter to follows the lumen of the oesophagus easily. Silicone also helps in increasing clarity and promoting maximum resilience of the device. The silicone gastroplasty tube has an exceptionally smooth surface which is produces by using a quality production technique. The silicone used in production of the tube is of 80 Shore A hardness so that it is sufficiently hard to prevent to occluding by twisting or compression.

The balloon 3 is designed to inflate asymmetrically. This has been found to produce a desirable anteriorly placed gastric pouch. The asymmetric balloon can be produced as follows:

1. A mould is produced which produces a balloon membrane that is of asymmetric thickness. It has a thin wall on one side and a thicker wall on the other.

2. The balloon is cast in soft ribbed silicone rubber of 30 Shore A hardness to produce controlled inflation. The balloon has a series of internal circumferential ribs and a smooth external surface. The ribbing prevents rupture and aids smooth surface texture.

3. The mould produces an orienting mark on the balloon so as the thin side of the balloon can be identified.

4. The balloon is fitted to the shaft of the tube so that the thin side of the balloon corresponds to the side on which the distance markings are imprinted on the shaft so that the eccentric inflation side of the balloon can be oriented anteriorly by the introducing operative.

The device must be inserted with care and force must never be used as the rigidity of the tube 4 may cause it to rupture the oesophagus. The tube should be well lubricated. The tip should be placed by the anaesthetist at the upper end of the oesophagus under vision using a laryngoscope. It can then be advanced down the oesophagus with safety. The tip can occasionally catch in the oesophagus at the cardio-oesophageal junction. In these cases the surgeon can often facilitate the passage of the device by applying pressure to the right side or posterior aspect of the junctional area with a laparoscopic instrument. This usually allows the tip to enter the stomach. The tube 4 should be advanced to the 50 cm mark before the positioning balloon 3 is inflated. This is to prevent damage to the oesophagus. Inflation of the balloon within the gastric lumen should be laproscopically observed by the surgeon to further ensure that no oesophageal trauma occurs. The balloon, once inflated, is drawn back against the cardio-oesophageal junction to provide an accurate indication of the correct placement position for the adjustable gastric band. If the tube will not pass readily the patient should be oesophagoscoped to exclude the presence of a stricture. Once the balloon is positioned against to cardio-oesophageal junction and the band placed beneath it the gastric band tunnelising sutures may be placed. As each of these sutures is inserted into the gastroplasty pouch the balloon should be deflated to avoid puncturing it. Once the suture is placed it can be reinflated.

Gastroplasty tube removal is generally simple. An empty syringe is attached to the balloon port 15, and the volume of air instilled is withdrawn. The tube is gently retracted. Occasionally, a balloon will fail to deflate, in which case, other interventions may be used. If the balloon fails to deflate at the end of the procedure, one step is to cut the tubing adjacent to the inflation valve to allow the balloon to deflate spontaneously. If this does not work, the area of obstruction is likely at the entrance to the inflation canal. The next manoeuvre is to cut the upper tubing below the point of entrance into the shaft of the device. If all else fails the balloon can be punctured through the gastric wall with a suture needle.

It is possible to suture the balloon to the gastric wall with one of the gastric band tunnelising sutures. In this case the balloon will deflate and when withdrawal is attempted the pouch will be tractioned upwards. The balloon will not be able to be removed without force. In this case it is necessary to attempt to identify the offending suture, remove and replace it.

There is a potential for perforating the stomach and/or the oesophagus. The tube is reasonably ridged and although it has a soft rounded flexible tip, use of injudicious force could cause perforation. If difficulty is experienced in passing the tube should be withdrawn and, as mentioned above, gastroscopy performed. Inflation of the calibrating balloon in the oesophagus could potentially cause trauma to the oesophagus, although this would be unusual with small volumes such as 20 ml of air. It is desirable however, as mentioned above, that the balloon is inflated intra-gastric to avoid this potential problem.

The foregoing described preferred forms of the present invention. Modifications, obvious to those skilled in the art can be made thereto without departing from the scope of the invention.

1. A gastric band positioning tubular apparatus comprising:
an extended tube having a series of lumens therein;
an asymmetrically inflating balloon located substantially at a distal end of the extended tube and interconnected with one of said series of lumens for the application of a positive air pressure thereto for asymmetric inflation of said balloon.

2. An apparatus as claimed in claim 1 wherein said extended tube includes a resilient flexible tip located at the distal end thereof.

3. An apparatus as claimed in claim 2 wherein said resilient flexible tip includes a first neck portion and a second bulbous end portion interconnected to said neck portion.

4. An apparatus as claimed in claim 1 wherein said tube includes a series of position markers along an exterior surface of the tube.

5. An apparatus as claimed in claim 4 wherein said asymmetrically inflated balloon is located so as to inflate asymmetrically relative to said position markers.

6. An apparatus as claimed in claim 1 further comprising a series of holes in said tube at the distal end thereof, said holes interconnected to a second suction lumen for sucking air and gastric juices out of a patient's stomach in use.

7. An apparatus as claimed in claim 1 wherein said extended tube is formed from substantially from silicone.

8. An apparatus as claimed in claim 7 wherein said silicone is substantially 89 Shore A hardness.

9. An apparatus as claimed in claim 1 wherein said balloon is formed from substantially 30 Shore A Hardness silicone.

10. An apparatus as claimed in claim 1 wherein said balloon includes a series of circumferential ribbed portions on an interior surface thereof.

11. An apparatus as claimed in claim 1 wherein said tube includes a series of exterior markings for circumferentially positioning said balloon on said extended tube.

12. An asymmetrically inflating balloon for use in gastric banding operations.

13. An asymmetrically inflating balloon as claimed in claim 12 wherein said balloon includes a series of circumferential ribbed portions on an interior surface thereof.

14. A method of placing a gastric band, the method including:
utilizing an asymmetrically inflating balloon in the stomach to position the gastric band around the stomach.

15. (canceled)

16. (canceled)
